(19) United States
(12) Patent Application Publication
(10) Pub. No.: US 2023/0390430 A1
(43) Pub. Date: Dec. 7, 2023

(54) METHOD AND APPARATUS FOR DISINFECTING A TUBE

(71) Applicant: Singletto Inc., Seattle, WA (US)

(72) Inventor: James Chongchu Chen, Clyde Hill, WA (US)

(21) Appl. No.: 18/205,082

(22) Filed: Jun. 2, 2023

Related U.S. Application Data
(60) Provisional application No. 63/348,838, filed on Jun. 3, 2022.

(51) Int. Cl. A61L 2/00

(52) U.S. Cl. CPC A61L 2/0094; A61L 2/0029; A61L 2202/24; A61L 2202/15; A61L 2202/11

(57) ABSTRACT

An applicator in the form of a container or cannister that is arranged to apply one or more photosensitizers to surfaces. The applicator includes at least one light source and the one or more photosensitizers. Light combined with the photosensitizers creates singlet oxygen and other radical species which are toxic to viruses and other pathogens. The photosensitizers may be combined in a powder form, or in an aqueous solution. Any light source can be used that emits the proper wavebands or wavelengths of light that are effectively absorbed by the photosensitizers leading to singlet oxygen generation.

### CROSS-REFERENCES TO RELATED APPLICATIONS

This application is a Utility Patent application based on previously filed U.S. Provisional Patent Application No. 63/348,838, filed on Jun. 3, 2022, the benefit of the filing date of which is hereby claimed under 35 U.S.C. § 119(e) and the contents of which is further incorporated in entirety by reference.

### BACKGROUND OF THE INVENTION

Historically, disinfection of medical devices, instruments, endoscopes, catheters, biopsy devices, and the like that contain small diameter channels or tubes can be problematic due to impaired access of disinfecting agents within the length of the tube or channel, and inadequate concentration and contact time of said agents in small diameter tubes or channels. Thus, there is a need for an improved way to provide disinfection of small diameter channels or tubes. For example, providing a more effective use of photosensitizers in solution, injected under pressure into a channel, illuminated by light sources proximate to a mouth of the channel which produces singlet oxygen and other reactive species achieving a useful rate and degree of antimicrobial activity and decontamination which prevents infections.

### DESCRIPTION OF VARIOUS EMBODIMENTS

The elements of one or more of the various embodiments are comprised of one or more photosensitizers, and one or more light sources emitting an overlapping light spectrum capable of photoactivating one or more photosensitizers. The photosensitizers are dissolved into an aqueous solution, preferably using sterile water, and injected into the mouth of the tube or channel using a nozzle which abuts the mouth of the tube or channel. The nozzle creates a fine aqueous mist that flows down the channel or tube, that contains at least one photosensitizer in solution. Along with the aqueous injection process, a bright light source is provided that directs light into the mouth of the tube or channel, illuminating the mist. The mist serves to refract, reflect, and/or diffuse light within the tube or channel creating a light pipe effect. The light that is transmitted down the tube serves to photoactivate the photosensitizer(s), which in turn generate singlet oxygen and other reactive oxygen species which inactivate or kill contaminating microorganisms within the tube or channel.

Alternatively, an aqueous fluid column is generated within the tube or channel which acts as a liquid light pipe, transmitting photoactivating light from a light source proximate to the orifice of the channel or tube, into the interior of the channel or tube, administered after instillation of at least one photosensitizer into the lumen of the tube or channel. A non-imaging lens with or without a mirror system proximate to the orifice of the tube or channel may be used as well, which serves to focus ambient light into the lumen of the tube or channel.

Also, one or more of the various embodiments comprise at least one photosensitizer formulation, at least one light source, and a photosensitizer applicator in the form of a container or cannister. Light combined with the photosensitizer creates singlet oxygen and other radical species which are toxic to viruses and other pathogens. All known and contemplated photosensitizers are intended to be part of this invention including but not limited to all types of methylene blue derivatives and methylene blue itself, xanthene dyes and derivatives, chlorophyll derivatives, tetrapyrrole structures, porphyrins, chlorins, bacteriochlorins, phthalocyanines, texaphyrins, prodrugs such as aminolevulinic acids, phenothiaziniums, squaraine, boron compounds, various transition metal complexes, hypericin, riboflavin, curcumin, titanium dioxide, psoralens, tetracyclines, flavins such as riboflavin, riboflavin derivatives, erythrosine, erythrosine derivatives, and the like. The most preferred photosensitizers are a combination of ones that are generally recognized as safe, and that are capable of absorbing light over a wide spectral range, such as erythrosine, methylene blue, and riboflavin. In one embodiment, the specific photosensitizers, methylene blue, erythrosine, and riboflavin are combined in a powder form, or in a aqueous solution. Concentrations range from 0.1 micromolar to 5,000 micromolar. A single photosensitizing agent can be used, or a combination of different photosensitizers are used.

One or more of a plurality of different types of light sources can be used that emits the proper wavebands or wavelengths of light that are effectively absorbed by the photosensitizers leading to singlet oxygen generation can be used. The illumination time and intensity of ambient light needed for adequate disinfection is determined empirically, experimentally, or derived from known data. The invention includes at least one light source comprised of light emitting diodes, xenon lamps, fluorescent bulbs and tubes, incandescent light bulbs, electroluminescent devices, lasers, and the like, even including natural sunlight. Other known or contemplated light sources are not excluded in any fashion, and include all known wavelengths and wavebands known to lead to a photodynamic effect particular to the photosensitizing agent. Light intensity ranges from 1 to lux, and is delivered for the duration of the decontamination process, which can range from one minute to 5 hours.

The photosensitizer(s) are supplied in a powder, a tablet, as granules, or in a pre-mixed aqueous solution, and are contained in a reservoir that can be pressurized using manual or automated compression. The aqueous solution is ejected through a nozzle which creates a mist that is propelled into the mouth of the channel or tube. At same time or prior to ejection of the mist into the channel or tube a light source is activated. The light source may utilize one or more lenses and/or mirrors that direct the light beam into the mouth of the channel or tube. The mist created by the nozzle is such that the droplet size, distribution, and density of the mist allows for light refraction, reflection, and diffusion within the length of the channel or tube, in a manner similar to the processes that are used to create cool light emitting diode water vapor fireplace effects. Since the mist contains at least one photosensitizer, light emitted from the mouth of the tube or channel and directed into the tube or channel using at least one lens, and/or mirror, and/or optical fiber photoactivates the photosensitizer(s) contained within the mist comprised of microdroplets.

The size range of microdroplets can range from 0.001 micron to 1000 microns, though it is understood that droplets can be produced below and above this range, and are included in the present disclosure.

Though an optical fiber could be inserted into the length of the tube or channel, delivering light at the orifice or mouth of the tube or channel is advantageous as it obviates an extra step of insertion, and minimizes inadvertent damage to the lumen of the tube or channel from the internal passage procedure. A bracket secured to a portion of a chamber or cabinet structure or platform on which, or in which the medical devices to be decontaminated are located can serve to direct water vapor and light delivery from the system or construct into the lumen of the tube or channel.

If a nozzle is used, the preferred type is the convergent type capable of creating and accelerating aqueous droplets containing one or more photosensitizer formulations of diameters ranging from submicron diameter to 1000 microns.

Alternatively, air containing one or more photosensitizer microdroplets is circulated into the tube or channel using high flow air circulation systems as is used in commercially available vaporized hydrogen peroxide cabinets used for high level disinfection of reusable medical equipment in healthcare settings.

In another embodiment, the microdroplets containing one or more photosensitizers are injected or circulated into the tube or channel first, then followed by illumination by the appropriate light spectrum and intensity using the light delivery construct which photoactivates the injected or circulating or transiting water vapor microdroplets within the lumen of the channel or tube.

In yet another embodiment, the illumination process enabled by the light delivery construct occurs simultaneously with injection or circulation of the microdroplets formulation containing one or more photosensitizers.

In one embodiment, light is aimed into the orifice of the tube or channel by an optical fiber capped with a microlens which focuses light into said orifice. Another embodiment uses at least one mirror which serves to collect and focus ambient light generated within the chamber or cabinet or which is present outdoors in order to focus light into the orifice of the tube or channel.

In another embodiment, the light delivery system is combined with the microdroplet generating device, which may use ultrasound and/or heat to generate the microdroplet vapor, which is injected under pressure by acceleration within a converging nozzle. The optimal density of the microdroplet stream and the rate of flow within the lumen of the tube or channel which transmits an effective total light dose is determined experimentally. Tubes or channels of various diameters and lengths, ranging from 1 mm to 1 cm or greater are preferentially tested with tube or channel lengths ranging from several cm to 200 cm or longer.

In yet another embodiment, a fluid column is generated within the tube or channel which acts as a light pipe. The fluid may contain one or more photosensitizers and can act as the light transmission means. In another embodiment an aqueous fluid containing at least one photosensitizer is injected into the mouth of the tube or channel, followed by illumination of said tube or channel, by directing and aiming light down the mouth of said tube or channel.

In one embodiment a mounting bracket directs and aims the light source towards the orifice(s) of the tube or channel.

In another embodiment, a non-imaging lens focuses ambient light into the orifice(s) of the tubes or channels. In addition, one or more mirrors may be used to capture and focus light into the non-imaging lens, which in turn launches light into the mouth of the channel or tube.

In another embodiment, the aqueous solution is subjected to ultrasound or heat creating the microdroplets, as occurs with the use of known steam generators using heat, or ultrasound.

In another embodiment, one or more photosensitizers are supplied as powders, granules, tablets, or in aqueous solution, in light proof containers, as part of a kit, which is also comprised of a portable, rechargeable light source such as a light emitting diode construct which is supplied with various lenses, enabling beam formation accommodating various tube or channel orifice apertures.

In another embodiment, one or more photosensitizers is dispensed from a spray container to the external surfaces of the devices and equipment to be decontaminated. An external light source in proximity to the devices and equipment to be decontaminated serves to photoactivate the photosensitizer sprayed onto the device and equipment surfaces. Lenses and or mirrors, may also be incorporated into the chamber, enclosure, or container holding the devices and equipment, that serve to focus and reflect light onto the surfaces to be decontaminated.

In another embodiment, supplemental oxygen is pumped or supplied to a container or chamber as part of the decontamination process, which is known to increase the singlet oxygen production from known type II photodynamic reactions. The optimal amount and rate of oxygen flow is determined experimentally, and is tested within a range of normal room air oxygen level to 100% oxygen air flow onto the container or chamber, at a rate of 1 L to 10 L per minute.

In another embodiment, a vacuum acting at the end of the tube or channel opposite where the fluid or microdroplet vapor is injected, is created within the container or chamber or cabinet which aids in aqueous fluid transfer by pulling fluid or microdroplet vapor from the distal end of the tube or channel.

In yet other embodiments, the tube is comprised of living tissue, such as the airway in a creature, or the urethra, or the gastrointestinal tract.

In other embodiments, the tube is comprised of an artificial material, such as may be found comprising a ureteral stent, an intravenous catheter, a ventriculostomy tube, an endotracheal tube, a gastrostomy tube, an arterial line, a central line, artificial or synthetic tubular grafts, and related tubes that exit from the body, enabling microdroplet delivery which can act as a type of light pipe, for light delivery. Light delivery may be therapeutic in itself, as is known in the phototherapy art, and/or used to activated at least one photosensitizer, delivered in the microdroplets, or previously administered. In these applications photosensitization can be used for disinfection purposes, to break down infectious biofilms, in addition, utilizing singlet oxygen and other reactive species to degrade and break down biological sediments such as proteins, blood clots, mucus, and the like which can cause a blockage of the tube.

In yet another embodiment, living tissue or synthetic tubes or grafts, such as those used as those used or functioning as blood vessels, are trans-cutaneously transilluminated after at least one photosensitizer is injected into said natural or synthetic blood vessels percutaneously.

In more embodiments, pipes or tubes that may be found in industrial, medical facilities, heating and cooling installations, and any sort of equipment with tubes or channels can be injected with microdroplet streams enabling light delivery enabling photodynamic disinfection, photodynamic polymerization of coatings and like to treat contamination, or seal leaks.

### DESCRIPTION OF FIGURES FOR VARIOUS EMBODIMENTS

FIG. 1A depicts an overview 100 of pressurized container 102 filled with one or more photosensitizer aqueous solution 104, with pressurized container 102 connected to flexible or rigid tube 106 which incorporates nozzle 108. Photosensitizer aqueous solution 104 is delivered to medical or equipment catheter, channel, or tube 110 via nozzle 108 which is positioned with an air gap, or directly juxtaposed to medical or equipment catheter, channel, or tube 110. Photosensitizer aqueous solution 104 is injected into tube 110 under pressure sufficient to fill tube 110 with photosensitizer aqueous solution 104. The orifice of nozzle 108 may fit within or be aligned with the mouth of tube 110, or be a 1.0 mm diameter or larger, up to 30.0 mm than the mouth of tube 110 which allows for simultaneous deposition of photosensitizer aqueous solution 104 within the lumen of tube 110 and extraluminal deposition of photosensitizer aqueous solution 104 on the external surface of tube 110. Photoactivation of photosensitizer aqueous solution 104 is accomplished by light source 112 emitting light waves (hr) that illuminate the external surface of tube 110 after surface deposition of photosensitizer aqueous solution 104.

FIG. 1B illustrates an overview 120 of an arrangement of light source 122 that incorporates optical fiber 124 which guides light waves 126 into the mouth of tube 128.

FIG. 1C shows an overview 130 of an arrangement of vapor mist generator 132 to generate cool aqueous steam 136 which is transmitted by hollow connector 134 and injected onto the lumen of tube 138, the cool steam or vapor serving to transmit light waves down the lumen of tube 138, which photoactivates photosensitizer aqueous solution 139 within the lumen of tube 138.

FIG. 1D shows an overview 140 of an arrangement of non-imaging lens 144 that is incorporated into an end of optical fiber 142 which focuses light waves 146 into an orifice of tube 146.

FIG. 2A depicts an overview 200 of an arrangement of chamber or cabinet or box 201 which incorporates elements described in FIGS. 1A-1D, enabling high level disinfection or decontamination of medical or other equipment J contained within chamber 201.

FIG. 2B shows an arrangement of intravenous bag 210 containing a medicinal fluid 212 connected to intravenous tubing 214.

FIG. 2C depicts an arrangement of intravenous tubing 214 that contains photosensitizer aqueous solution 216 which is photoactivated by light source 218, which illuminates photosensitizer solution 216 through optically transparent intravenous tubing 214. In all cases of illumination of photosensitizer aqueous solutions, the photoactivation process is such that pathogens are inactivated or killed.

Also, FIG. 2C depicts an arrangement of urinary catheter 222 connected to urine drainage collection bag 224. Urinary catheter 222 is shown exiting urethral orifice 220. External light source 226 emits light waves (hr) that illuminate photosensitizer aqueous solution 228 contained in the lumen of urinary catheter 222 and bag 224, killing or inactivating pathogens within the optically transparent urinary catheter 222 and/or within optically transparent urine collection bag 224.

FIG. 3 shows an overview 300 of an arrangement of mirror system 302 that employs mirrors 306A, 306B and 306C to direct photoactivating light waves (hr) emitted from light source 304 into the orifice of tube 308.

FIG. 4A illustrates an arrangement of pump 400 which pumps steam, vapor, or liquid optionally containing at least one photosensitizer formulation into the orifice 404 of rigid or flexible polymeric or metallic hose 402. Also shown is reservoir 406 which holds and supplies at least one photosensitizer formulation in an aqueous solution form or a powder form which is delivered into the orifice 404.

FIG. 4B shows an arrangement of adjustable clamp 412 which surrounds hose 410 which is positioned around the mouth orifice 416 of tube or channel 414 which may optionally represent the distal or proximal end of an endoscope or a pipe.

FIG. 4C illustrates an arrangement of tub or container 420 which is shown containing or incorporating pump assembly 422 which also contains a vapor, steam, or liquid with or without at least one photosensitizer formulation. Pump 422 is connected to tube or hose 424 which is connected reversibly to tube or pipe 426.

FIG. 4D shows an arrangement of light emitting diode (LED) array 430 surrounding the inlet 432 of tube, pipe, or channel 434. LED array 430 emits light waves towards the origin of tube, pipe or channel 434, causing illumination throughout the length of tube or pipe or channel 434. In FIGS. 4A, 4B, 4C and 4D, the arrangements enable at least one photosensitizer formulation to be delivered and transmitted along the length of tubes and/or channels, which may represent endoscopes, catheters, vascular grafts, stents, hoses, pipes, and the like, which are in need of decontamination.

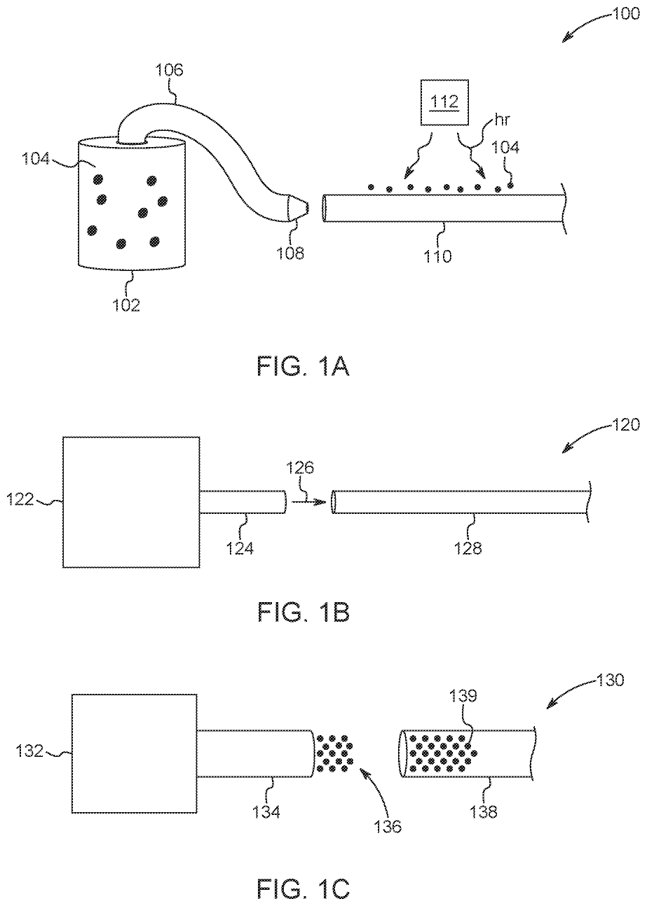

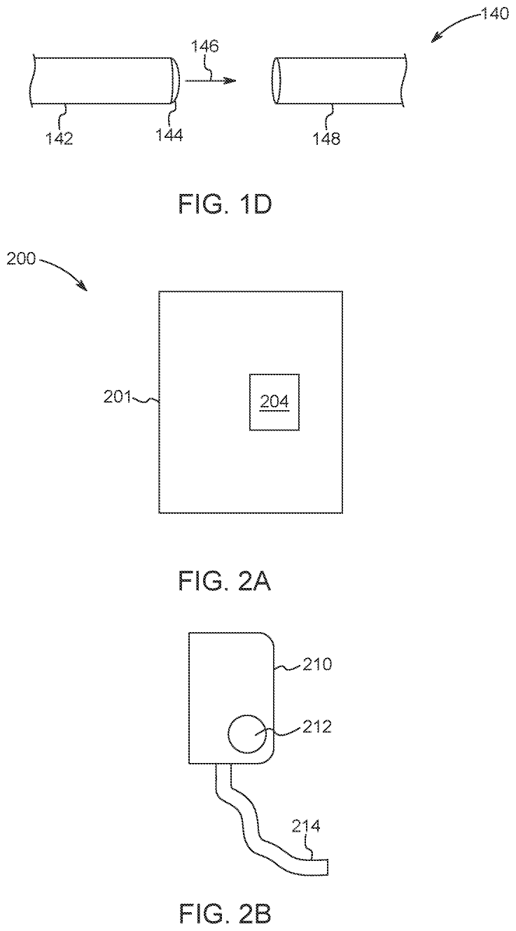

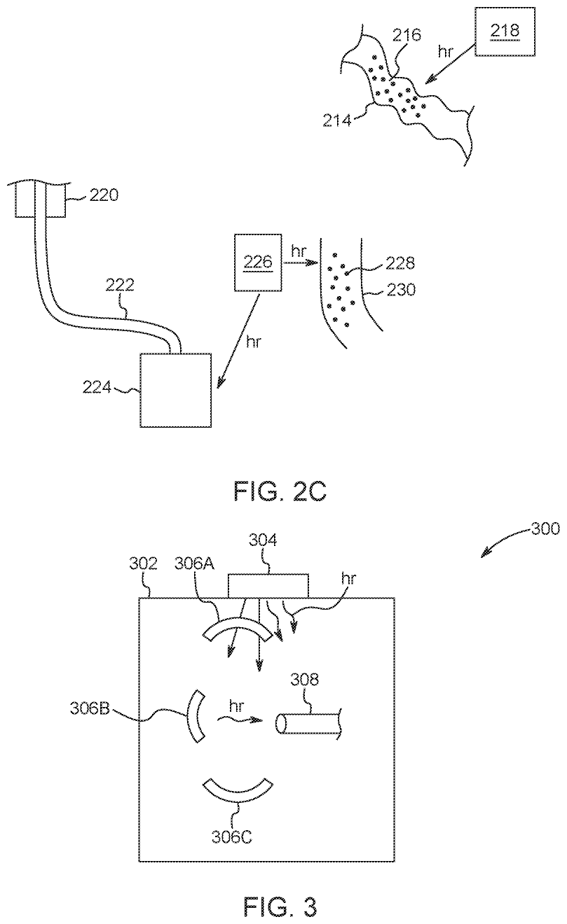

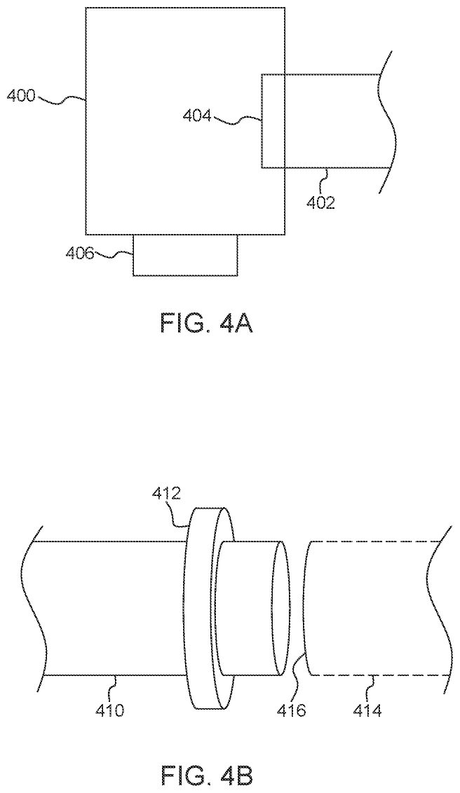

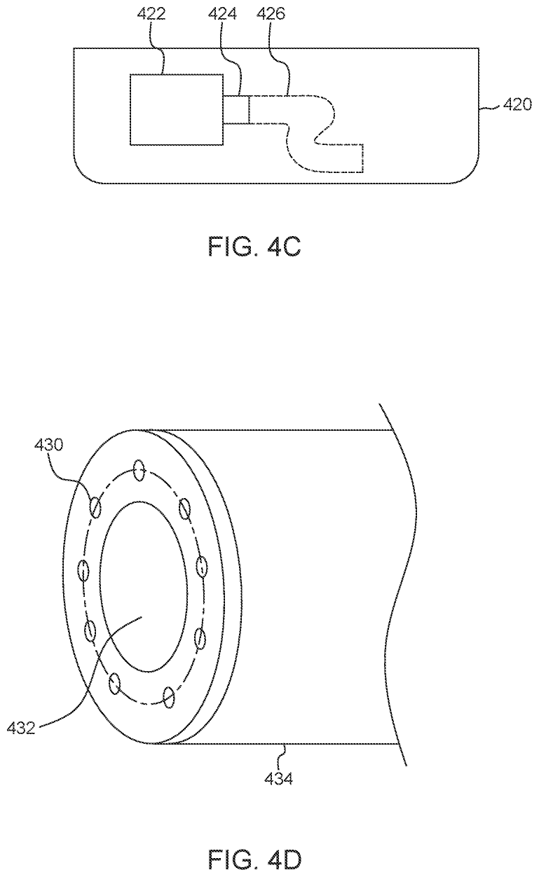

Furthermore, in one or more embodiments, a photosensitizer formulation may be activated via vapor, steam, or an optically transparent liquid capable of transmitting light in a similar fashion to a light pipe, or cool steam fireplace, wherein the emitted light waves are of sufficient intensity and spectral overlap to enable useful and effective photoactivation of a photosensitizer formulation.

### DESCRIPTION OF EXAMPLES FOR VARIOUS EMBODIMENTS

### Example 1

A series of laboratory experiments may be carried out using tubes of various diameters and lengths containing vapor microdroplets of varying diameters, through which light is delivered at the orifices of the tubes. Maximum light transmission is measured at the ends of the tubes opposite the orifice, in order to determine the optimum range of vapor microdroplet sizes and microdroplet density that transmits an effective total light dose, which activates at least one photosensitizer. In addition, various combinations and concentrations of photosensitizers are tested for pathogen and toxin inactivation and degradation at various light transmission parameters. Speed and degree of pathogen and toxin inactivation and degradation are determined to select the optimal the photosensitizer and light conditions, which includes a determination of optimal vapor microdroplet speed and flow in the various tube diameters, lengths, and configurations. Configurations may include linear straight disposition of the tubes, with or without coiling and bending configurations. Test pathogens and toxins are inoculated into the tubes at various distances, and in various amounts for inactivation and killing tests. In these experiments, the microdroplets may contain at least one photosensitizer formulation in an aqueous solution, with the microdroplets serving a dual purpose of light transmission and photoactivation for decontamination purposes.

### Example 2

Another experiment may include a balloon tipped urinary catheter that is decontaminated using a system comprising an oral riboflavin formulation and a methylene blue formulation which is contained within the positioning balloon which is part of the urinary catheter, as in the well-known Foley urinary catheter. The methylene blue formulation is injected through the balloon fluid filling port into the reservoir balloon, which incorporates a membrane interface with the drainage catheter which allows for passage of a metered amount of methylene blue solution into the urine drainage part of the urinary catheter. Photoactivating light, which in the case of methylene blue and riboflavin are centered on the red and blue absorption bands respectively, is supplied by a light source, which may be a light emitting diode array located external to the distal urethral orifice. The light source transmits light from a distal to proximal direction, causing a pathogen inactivation and killing effect within the drainage catheter. An option is bidirectional light delivery, simultaneously into the urinary drainage channel and the drainage collection bag. The oral riboflavin will be excreted into the urinary tract, eventually into the bladder. With blue light directed into the bladder, pathogens in the bladder can be inactivated and killed. In addition, if the positioning balloon is temporarily partially deflated or positioned deeper into the bladder, which allows for leakage of riboflavin containing urine around the exterior of the catheter, pathogens external to the catheter wall can be inactivated and killed by light transmitted through the wall of the catheter, which in this case is comprised of an optically transparent polymer.

### Example 3

Another experiment may be performed during the precleaning and/manual cleaning stage of a high level endoscope disinfection, an aqueous solution containing at least one photosensitizer such as methylene blue and/or riboflavin, at concentrations ranging from 10 to 1000 micromolar are injected from a polymeric catheter into the proximal mouths of one or more endoscope channels. The injection process occurs till the aqueous fluid is visualized at the distal end. Light is then delivered, red light for methylene blue and blue light for riboflavin into the distal and/or proximal ends of the endoscope channels, photoactivating the photosensitizer(s) for antimicrobial effect, which includes biofilm eradication.

What is claimed as new and desired to be protected by Letters Patent of the United States is:

1. A device, comprising:
a photosensitizer formulation; and
an elongated structure with a lumen that is configured for medical use, wherein illumination of the photosensitizer formulation causes production of singlet oxygen inside the lumen, and wherein the singlet oxygen causes antimicrobial activity to decontaminate the lumen to prevent microbial infection of a patient.

2. The device of claim 1, wherein the elongated structure, further comprises:
one or more of a tube or a channel that is formed by one or more of an intravenous tube, a medical instrument, an endoscope, a catheter, or a biopsy device.

3. The device of claim 1, further comprising:
one or more light sources that are configured to illuminate the photosensitizer formulation to cause the production of singlet oxygen, wherein the one or more light sources include one or more a light emitting diode, xenon lamp, fluorescent bulb or tube, incandescent light bulb, electroluminescent device, lasers, or natural sunlight.

4. The device of claim 1, further comprising:
one or more light sources that are configured to illuminate the photosensitizer formulation, wherein a light intensity of the illumination ranges from 1 to 50,000 lux, and wherein one or more portions of the light sources are arranged remotely to the elongated structure, proximate to an orifice of the lumen or integrated within the elongated structure to provide illumination of the photosensitizer formulation inside the lumen.

5. The device of claim 1, wherein the decontamination further comprises:
employing the antimicrobial activity caused by the singlet oxygen to decontaminate the lumen for a period of time that ranges from one minute to 5 hours.

6. The device of claim 1, wherein the elongated structure, further comprises:
one or more portions of the elongated structure that are optically transparent or translucent, wherein one or more light sources are configured to emit light proximate to one or more of an orifice of the lumen or through the one or more portions to illuminate the photosensitizer formulation.

7. The device of claim 1, wherein the photosensitizer formulation, further comprises:
a fluid that is dissolved with the photosensitizer formulation to form an aqueous solution, wherein the aqueous solution is atomized into a mist of droplets that flows along a length of the lumen of the elongated structure, wherein the droplets provide one or more of refraction, reflection or diffusion of light transmitted within the length of the lumen.

8. The device of claim 1, further comprising:
a container for a fluid that is dissolved with the photosensitizer formulation to form an aqueous solution; and
a connector that is configured to couple an outlet of the container to the lumen of the elongated structure, wherein the aqueous solution flows from the outlet of the container along a length of the lumen.

9. The device of claim 1, wherein the photosensitizer formulation, further comprising:
one or more of methylene blue or riboflavin at a concentration having a range from 10 to 1000 micromolar.

10. The device of claim 1, further comprising:
an applicator that is configured for providing the photosensitizer formulation into an orifice of the lumen within the elongated structure.

11. The device of claim 1, further comprises:
a pump that is coupled to a container for a fluid that is dissolved with the photosensitizer formulation to form an aqueous solution, wherein the pump is configured to pressurize the aqueous solution into an orifice of the lumen to create a flow along a length of the lumen.

12. The device of claim 11, wherein the pump further comprises:
a nozzle to convert the aqueous solution into a mist of droplets directed into the orifice of the lumen to spread the mist of droplets along the length of the lumen.

13. The device of claim 1, wherein the photosensitizer formulation further comprises:
a mixture of photosensitizer formulation particles that provide a powder form for the photosensitizer formulation.

14. A method, comprising:
providing a photosensitizer formulation; and
employing an elongated structure with a lumen that is configured for medical use to illuminate the photosensitizer formulation to cause production of singlet oxygen inside the lumen, wherein the singlet oxygen causes antimicrobial activity to decontaminate the lumen to prevent microbial infection of a patient.

15. The method of claim 14, wherein the elongated structure, further comprises:
providing one or more of a tube or a channel that is formed by one or more of an intravenous tube, a medical instrument, an endoscope, a catheter, or a biopsy device.

16. The method of claim 14, further comprising:
providing one or more light sources that are configured to illuminate the photosensitizer formulation to cause the production of singlet oxygen, wherein the one or more light sources include one or more a light emitting diode, xenon lamp, fluorescent bulb or tube, incandescent light bulb, electroluminescent device, lasers, or natural sunlight.

17. The method of claim 14, further comprising:
providing one or more light sources that are configured to illuminate the photosensitizer formulation, wherein a light intensity of the illumination ranges from 1 to 50,000 lux, and wherein one or more portions of the light sources are arranged remotely to the elongated structure, proximate to an orifice of the lumen or integrated within the elongated structure to provide illumination of the photosensitizer formulation inside the lumen.

18. The method of claim 14, wherein the decontamination further comprises:
employing the antimicrobial activity caused by the singlet oxygen to decontaminate the lumen for a period of time that ranges from one minute to 5 hours.

19. The method of claim 14, wherein the elongated structure, further comprises:
providing one or more portions of the elongated structure that are optically transparent or translucent, wherein one or more light sources are configured to emit light proximate to one or more of an orifice of the lumen or through the one or more portions to illuminate the photosensitizer formulation.

20. The method of claim 14, wherein the photosensitizer formulation, further comprises:
providing a fluid that is dissolved with the photosensitizer formulation to form an aqueous solution, wherein the aqueous solution is atomized into a mist of droplets that flows along a length of the lumen of the elongated structure, wherein the droplets provide one or more of refraction, reflection or diffusion of light transmitted within the length of the lumen.
